(19) United States
(12) Patent Application Publication
(10) Pub. No.: US 2026/0301228 A1
(43) Pub. Date: Oct. 1, 2026

(54) METHOD FOR COLOR CORRECTION BASED ON GROUND TRUTH COLOR CHART

(71) Applicant: Purdue Research Foundation, West Lafayette, IN (US)

(72) Inventors: Young KIM, West Lafayette, IN (US); Sang Mok PARK, West Lafayette, IN (US); Yuhyun JI, West Lafayette, IN (US); Semin KWEON, West Lafayette, IN (US); Jungwoo LEEM, West Lafayette, IN (US)

(73) Assignee: Purdue Research Foundation, West Lafayette, IN (US)

(21) Appl. No.: 19/489,831

(22) PCT Filed: Apr. 20, 2024
(86) PCT No.: PCT/US2024/025609

§ 371 Date: Dec. 3, 2025

Related U.S. Application Data
(60) Provisional application No. 63/524,318, filed on Jun. 30, 2023.
(60) Provisional application No. 63/524,352, filed on Jun. 30, 2023.

(51) Int. Cl. G06T 7/90; G06T 7/00; G06T 11/10

(52) U.S. Cl. CPC G06T 7/90; G06T 7/0012; G06T 11/10; G06T 2207/10024; G06T 2207/20081; G06T 2207/20084; G06T 2207/30036; G06T 2207/30041; G06T 2207/30088; G06T 2210/41

(57) ABSTRACT

A method for correcting colors in a captured image, which includes receiving a ground truth color chart having a plurality of reference color patches, capturing an image based on a first color domain from a scene or sample of interest juxtaposed with the ground truth color chart, obtaining ground truth color values associated with each of the reference color patches in a second color domain, generating a scene or sample-specific dataset using the plurality of color values of the reference color patches in the ground truth color chart in the first and second color domains as the input and output, training a neural network of color correction with the dataset, and applying the trained neural network to convert portion of the scene or sample of interest outside the ground truth color chart in the first color domain to thereby generate an output image based on the second color domain.

### CROSS-REFERENCE TO RELATED APPLICATIONS

The present non-provisional patent application is related to and claims the priority benefit of U.S. Provisional Patent Application Ser. 63/524,318, filed Jun. 30, 2023, and of U.S. Provisional Patent Application Ser. 63/524,352, filed Jun. 30, 2023, the contents of each of which are hereby incorporated by reference in their entirety into the present disclosure.

### STATEMENT REGARDING GOVERNMENT FUNDING

This invention was made with government support under R01EB033788 awarded by the National Institutes of Health. The government has certain rights in the invention.

### TECHNICAL FIELD

The present disclosure generally relates to color correction in bioimaging and in particular to color correction based on a predetermined ground truth color chart, and further determining concentration of a molecule in a sample based on the color corrected bioimaging.

### BACKGROUND

This section introduces aspects that may help facilitate a better understanding of the disclosure. Accordingly, these statements are to be read in this light and are not to be understood as admissions about what is or is not prior art.

A photograph can provide diagnostic information beyond a mere visual representation. The primary challenge is to achieve color accuracy, which is referred to as the ability to define standardized colors of a sample and accurately detect them across diverse photo acquisition settings. Photographs acquired using a digital camera exhibit detrimental variations in colors, depending on device models, light conditions, and file formats. Color accuracy in medical imaging, color consistency in machine vision, and color constancy in human perception are distinct yet interrelated aspects of color science and management. The importance of color consistency is well recognized in machine vision and general photography, which focuses on color correction and color reproduction. Color constancy in human perception primarily focuses on achieving identical perceptual response. However, there is a lack of studies to realize color accuracy in medical imaging. Recently, the onboard cameras of mobile devices have been extensively used for medical photography, including telemedicine and mobile health (mHealth) applications. Healthcare professionals now consider the acquisition of photos using mobile devices (i.e., smartphones and tablets) to be a mandatory part of healthcare practice because diagnostic photography plays a key role in a variety of digital health applications. As a result, there is an urgent need for implementing color accuracy in diagnostic photography.

Reproducing original colors is challenging in photography, printing, and electronic displays because of intrinsic color distortions during image acquisition, processing, and output. The existing color calibration or correction methods using the conventional color charts are inadequate to achieve high fidelity of color accuracy for diagnostic purposes. First, generating a universal color gamut that can exactly reproduce all colors is challenging. Optimizing a unique color gamut is necessary, but identifying relevant yet subtle color derivatives is not straightforward without spectroscopic analyses, which enable to augment homogeneous data for ideal gamut determination. Second, color undersampling in the conventional color charts fundamentally reduces the degree of color accuracy. Color correction and calibration computations with a limited number of reference colors are prone to random and systematic errors (e.g., Gaussian, impulse, photon, or speckle noise) in a variety of photo acquisitions. Incorporating more reference colors allows us to use advanced machine learning techniques that can outperform the conventional computations. Third, regression-based color correction computations (also known as lookup table and interpolation) commonly used in machine vision require manual fine-tuning, depending on photo acquisition settings.

In digital photography, color appearance is considerably affected by a variety of factors, settings, and conditions. Reference is made to FIG. 1A wherein representative digital photos of biological tissue (e.g., palpebral conjunctiva) captured under four various white-light illumination conditions are provided including LEDs with color temperature of 3000 K, 4300 K, and 5800 K as well as fluorescent tube light. Color accuracy in diagnostic photography refers to the ability to detect the absolute colors across various photo acquisition settings. From a device standpoint, a digital trichromatic camera (three-color image sensor) has unique red-green-blue (RGB) spectral response functions (also known as the spectral sensitivity) as a function of the wavelength of light A. Referring to FIG. 1B, model-specific RGB spectral response functions lead to device-dependent color mapping of several popular low- and high-end smartphones with ANDROID and iOS operating systems including APPLE IPHONE 12 PRO, APPLE IPHONE SE, SAMSUNG GALAXY S21, and SAMSUNG GALAXY A52. Notably, the RGB spectral responses of smartphone cameras exhibit significant model-specific variations as shown in FIGS. 1C and 1D, resulting in notoriously device-dependent RGB color values. From an illumination standpoint, different types of white light sources have distinct spectral profiles in otherwise grossly white-appearing light conditions as shown in FIGS. 1E and 1F. Diverse spectral characteristics of light sources fundamentally limit color management (e.g., white balancing), resulting in light condition-specific RGB color values as shown in FIG. 1G. From file format and bit depth (color depth) standpoints, the degree of color compression is significantly influenced by the image file format as shown in FIGS. 1H and 1I. JPEG, the most commonly used file format (8-bit depth), uses lossy compression to reduce file size. RAW (also known as DNG) minimizes data compression and rendering. Recent high-end smartphones provide Pro Mode or ProRAW for 10-bit depth in each RGB channel. In telemedicine settings (FIG. 1H), live, two-way video conferencing between a healthcare provider and patient introduces additional diversity in file formats (e.g., MP4). Overall, diverse photo acquisition scenarios pose a fundamental challenge for color accuracy in diagnostic photography.

Color correction is critical when the images are being used for diagnostic purposes. For example, suppose images are acquired from a biological tissue and those images are being used to make diagnostic decisions. Without color correction, wrong diagnostic decisions may be made that are detrimental to the outcome of the diagnostic process.

The primary drawback of regression-based color corrections (lookup table and interpolation) is their limited adaptability across diverse photo acquisition settings, resulting in a lack of generalizability. To enhance color correction performance beyond the simple 3×3 linear color correction, the higher-degree RGB polynomial (or root-polynomial) expansion terms are frequently incorporated. However, the impact of polynomial expansions on color correction is scene/sample-specific, and their effectiveness is not consistently guaranteed. Consequently, an appropriate set of polynomial expansions must be selected manually and empirically for each photo acquisition.

Neural network-based color recovery can overcome this intrinsic limitation. A neural network architecture allows the incorporation of possible polynomial expansion terms, and the weights of the connections between the nodes across the layers of the network can be determined specifically for a given scene or sample. After scene/sample-specific training, the algorithm takes the RGB values acquired from each pixel as the input and returns the CIE XYZ values under CIE illuminant E as the output.

Specifically, a neural network algorithm enables fully automated and high-fidelity color recovery under diverse acquisition scenarios. The reference colors in the color chart serve as training data for a particular scene or sample in which the chart is juxtaposed with the sample of interest. Importantly, a neural network-based color recovery algorithm has three advantages. First, given a relatively small amount of training data, the proposed network is concise and consists of only three layers: input, hidden, and output layers. Second, sigmoid activation functions are employed to be highly suitable for shallow networks, supported by the universal approximation theory. Third, the hidden layer mimics all possible terms of the RGB polynomial (root-polynomial) expansions. This hidden layer design completely avoids the manual selection of appropriate expansion terms for a specific scene or sample.

The neural network algorithm can recover absolute colors under CIE illuminant E with high fidelity in a fully automated manner. The absolute CIE XYZ (under CIE illuminant E) and acquired RGB values of the reference colors in the color chart served as a training dataset for the neural network, which was specific to a photo.

Importantly, the network architecture mimicked regression-based color corrections that include higher-degree RGB polynomial expansions. For example, the hidden layer can capture the nonlinearity between the CIE XYZ and RGB values with 29 nodes, which are equivalent to all possible 29 terms of RGB polynomial expansions. The input layer has three nodes corresponding to the RGB values acquired using a digital camera (x_(3×1)=[R,G,B]\hat{ }T), whereas the output layer with three nodes returns the recovered CIE XYZ values (y_(3×1)=[X,Y,Z]\hat{ }T) under a specific photo acquisition setting. Batch normalization is applied to the hidden layer for efficient and accurate training. A sigmoid activation function is used after batch normalization. The sigmoid function exhibits superior performance compared to other activation functions in shallow neural networks, as supported by the universal approximation theory. The network is trained with the root mean square error over a maximum of 500 epochs. The Adam optimizer with an initial learning rate of 5×10-4 and a mini-batch size of 30 is used.

Therefore, there is an unmet need for a novel method that can use a ground truth color chart and correct colors in captured images to true colors.

### SUMMARY

A method for correcting colors in a captured image is disclosed. The method includes receiving a ground truth color chart having a plurality of reference color patches, capturing an image based on a first color domain from a scene or sample of interest juxtaposed with the received ground truth color chart, the captured image thereby including the plurality of reference color patches, obtaining ground truth color values associated with each of the reference color patches in a second color domain, generating a scene or sample-specific dataset using the plurality of color values of the reference color patches in the ground truth color chart in the first color domain and the second color domain as the input and output, respectively, training a neural network of color correction with the scene or sample-specific dataset, and applying the trained neural network to convert portion of the scene or sample of interest outside the ground truth color chart in the captured image in the first color domain to thereby generate an output image based on the second color domain.

In the above method, the first color domain is the same as the second color domain.

In the above method, the first color domain is different than the second color domain.

In the above method, the ground truth color values based on the second color domain associated with each of the reference color patches are defined under International Commission on Illumination (CIE) illuminant E.

In the above method, spatial nonuniformity of illumination is corrected in the capture image.

In the above method, the scene or sample of interest is from a biological tissue.

In the above method, the biological tissue includes palpebral conjunctiva.

In the above method, the biological tissue includes skin.

In the above method, the biological tissue includes teeth.

In the above method, the biological tissue includes tongue.

In the above method, the biological tissue includes eye.

In the above method, the biological tissue includes bulbar conjunctiva.

In the above method, the biological tissue includes urine.

In the above method, the biological tissue includes whole blood.

In the above method, the biological tissue includes eye tears.

In the above method, the biological tissue includes nailbed.

In the above method, the biological tissue includes external eye.

In the above method, the biological tissue includes external saliva.

In the above method, the biological tissue includes external stool.

In the above method, the biological tissue includes external body fluid.

In the above method, the biological tissue includes biological solutions.

In the above method, each of the first or second color domains includes RGB.

In the above method, each of the first or second color domains includes CIE XYZ.

In the above method, each of the first or second color domains includes CIE YUV.

In the above method, each of the first or second color domains includes CIE UVW.

In the above method, each of the first or second color domains includes CIE RGB.

In the above method, each of the first or second color domains includes CIE LAB.

In the above method, each of the first or second color domains includes CIE LUV.

In the above method, each of the first or second color domains includes HSL.

In the above method, each of the first or second color domains includes HSV.

In the above method, each of the first or second color domains includes HCL.

In the above method, each of the first or second color domains includes LMS.

In the above method, the neural network is a multilayer perceptron.

In the above method, the neural network includes one or more hidden layers which employ one of sigmoid activation function or sigmoid linear unit activation function.

In the above method, the neural network includes connections defined by weights which weights are initialized by sampling from a predetermined distribution.

Another method of determining concentration of a molecule in a sample from a captured image is disclosed. The method includes establishing a color-corrected image (output image) from a captured image from a scene or sample of interest, the output image provides color corrections from a first color domain to a second color domain, the established output image associated with color values of the sample in the second color domain, and using color values of the output image of the sample in the second color domain to determine the concentration of a molecule of interest.

The above method further includes determining full spectral values from the associated color values of the output image in the second color domain, when a priori spectral data are available.

In the above method, the full spectral values are calculated from

B_{N×r}=R_{N×p}A_{p×r},
wherein N represents number of wavelengths of spectral values based on a predetermined resolution,
r represents number of a priori spectral data,
R_{N×p} represents a transformation matrix to transform the determined color values of the output image in the second color domain to full spectral values, where p represents number of polynomial expansion terms, and
A_{p×r} represents an expanded matrix of the determined color values of the output image in the second color domain represented by:

$${A}_{p \times r} = {[ \begin{matrix}{D}_{{21}_{1}} & {D}_{2 {2}_{1}} & {D}_{2 {3}_{1}} & ⋯ & {D}_{2 {1}_{1}}^{i} {D}_{2 {2}_{1}}^{j} {D}_{2 {3}_{1}}^{k} & \sqrt[i + j + k]{{D}_{{21}_{1}}^{i} {D}_{2 {2}_{1}}^{j} {D}_{2 {3}_{1}}^{k}} \\ ⋮ & ⋮ & ⋮ & ⋮ & ⋮ & ⋮ \\ {D}_{2 {1}_{r}} & {D}_{2 {2}_{r}} & {D}_{2 {3}_{r}} & … & {D}_{{21}_{r}^{i}} {D}_{2 {2}_{r}}^{j} {D}_{2 {3}_{r}}^{k} & \sqrt[i + j + k]{{D}_{21 r}^{i} {D}_{22 r}^{j} {D}_{2 {3}_{r}}^{k}}\end{matrix} ]}^{T}$$

wherein i, j, and k represent degree of expansion,
D₂₁ represents first color value of the second color domain,
D₂₂ represents second color value of the second color domain,
and D₂₃ represents third color value of the second color domain.

In the above method, the determined concentration of the molecule of interest establishes blood hemoglobin.

In the above method, blood hemoglobin concentration is determined based on the calculated full spectral values and a spectrum-to-concentration model as compared to a predetermined population spectral values and associated population blood hemoglobin concentration values using partial least squares regression.

In the above method, the spectrum-to-concentration model is:

$$Hgb = {c}_{0} + {c}_{1} I ( {λ}_{1} ) + {c}_{2} I ( {λ}_{2} ) + {c}_{3} I ( {λ}_{3} ) … + {c}_{N} I ( {λ}_{N} )$$

wherein Hgb represents blood hemoglobin concentration, and
coefficients c₀, c₁, c₂, c₃, . . . , c_{N} are determined using a priori subject population blood hemoglobin concentration data vs. spectral intensity data across a range of wavelengths (λ₁, λ₂, λ₃, . . . , λ_{N}).

The above method further includes receiving a color-to-concentration model expressing concentration of the molecule based on colors in the second color domain, and applying the color-to-concentration model to the output image to thereby obtain concentration of the molecule from the output image using least squares regression.

In the above method, the color-concentration model is:

$$Concentration = {c}_{0} + {c}_{1} {D}_{21} + {c}_{2} {D}_{22} + {c}_{3} {D}_{23} … + {c}_{p - 1} {D}_{21}^{i} {D}_{22}^{j} {D}_{23}^{k} + {c}_{p} \sqrt[i + j + k]{{D}_{21}^{i} {D}_{22}^{j} {D}_{23}^{k}}$$

wherein i, j, and k represent degree of expansion,
D₂₁ represents the first color value of the second color domain,
D₂₂ represents the second color value of the second color domain,
D₂₃ represents the third color value of the second color domain,
p represents number of polynomial expansion terms, and
coefficients c₀, c₁, c₂, c₃, . . . , c_{N} are determined using a priori subject population molecule concentration data vs. color values in the second color domain.

In the above method, the first color domain is the same as the second color domain.

In the above method, the first color domain is different than the second color domain.

In the above method, the second color domain is defined under International Commission on Illumination (CIE) illuminant E.

In the above method, the scene or sample of interest is from a biological tissue.

In the above method, the biological tissue includes palpebral conjunctiva.

In the above method, the biological tissue includes skin.

In the above method, the biological tissue includes teeth.

In the above method, the biological tissue includes tongue.

In the above method, the biological tissue includes eye.

In the above method, the biological tissue includes bulbar conjunctiva.

In the above method, the biological tissue includes urine.

In the above method, the biological tissue includes whole blood.

In the above method, the biological tissue includes eye tears.

In the above method, the biological tissue includes nailbed.

In the above method, the biological tissue includes external eye.

In the above method, the biological tissue includes external saliva.

In the above method, the biological tissue includes external stool.

In the above method, the biological tissue includes external body fluid.

In the above method, the biological tissue includes biological solutions.

In the above method, each of the first or second color domains includes RGB.

In the above method, each of the first or second color domains includes CIE XYZ.

In the above method, each of the first or second color domains includes CIE YUV.

In the above method, each of the first or second color domains includes CIE UVW.

In the above method, each of the first or second color domains includes CIE RGB.

In the above method, each of the first or second color domains includes CIE LAB.

In the above method, each of the first or second color domains includes CIE LUV.

In the above method, each of the first or second color domains includes HSL.

In the above method, each of the first or second color domains includes HSV.

In the above method, each of the first or second color domains includes HCL.

In the above method, each of the first or second color domains includes LMS.

### BRIEF DESCRIPTION OF FIGURES

FIG. 1A provides representative digital photos of biological tissue (e.g., palpebral conjunctiva) captured under five various white-light illumination conditions are provided including LEDs with color temperature of 3000 K, 4300 K, and 5800 K as well as fluorescent tube light.

FIG. 1B provides model-specific RGB spectral response functions which lead to device-dependent color mapping of several popular low- and high-end smartphones with ANDROID and iOS operating systems including APPLE IPHONE 12 PRO, APPLE IPHONE SE, SAMSUNG GALAXY S21, and SAMSUNG GALAXY A52.

FIGS. 1C and 1D provide device-dependent RGB color values.

FIGS. 1E and 1F provides different types of white light sources which have distinct spectral profiles in otherwise grossly white-appearing light conditions.

FIG. 1G provides diverse spectral characteristics of light sources which fundamentally limit color management (e.g., white balancing), resulting in light condition-specific RGB color values.

FIGS. 1H and 1I provide degree of color compression (FIG. 1H: schematic of file compressions) significantly influenced by the image file format (FIG. 1I: DNG, JPEG, and MP4).

FIG. 2 is a flowchart that describes the methodology of the present disclosure at a high level.

FIG. 3 is an example of a ground truth color chart.

FIG. 4 is a flowchart depicting alternative processes for determining concentration from a corrected image, according to the present disclosure.

FIG. 5 is an example of example of a scene of interest with the ground truth color chart of FIG. 3 juxtaposed therein.

FIG. 6 provides blood hemoglobin quantification results using the ground truth color chart of FIG. 5.

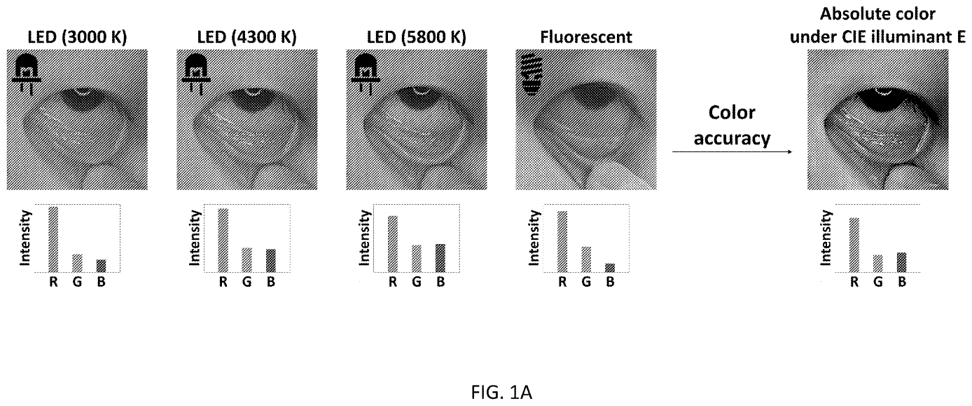

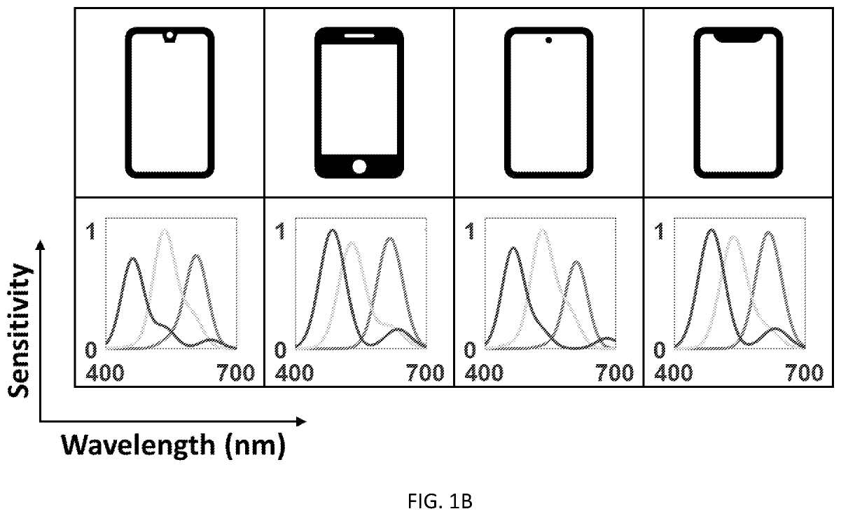

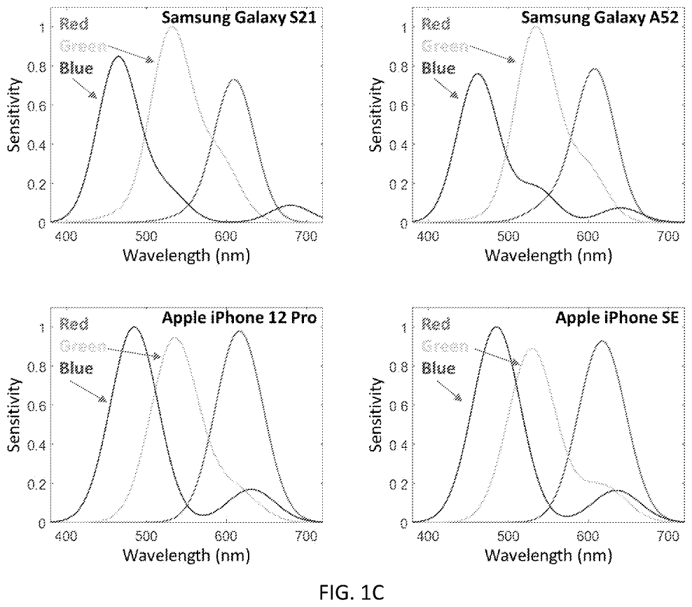

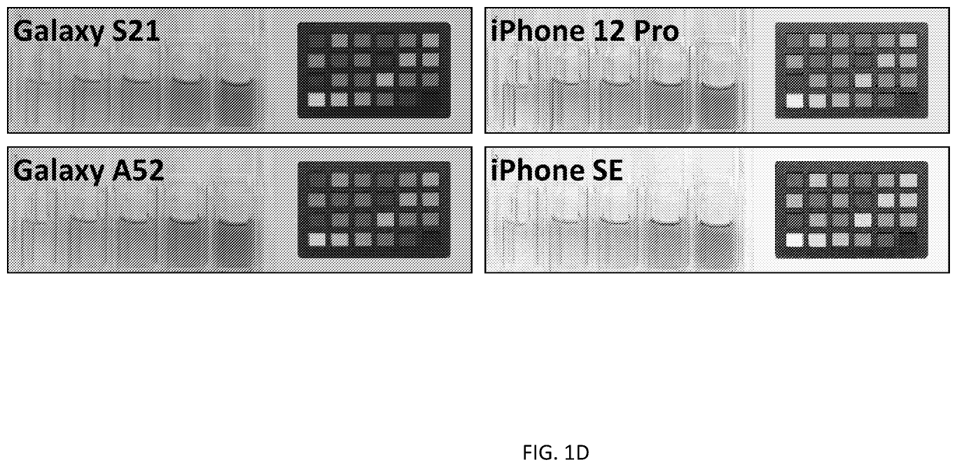

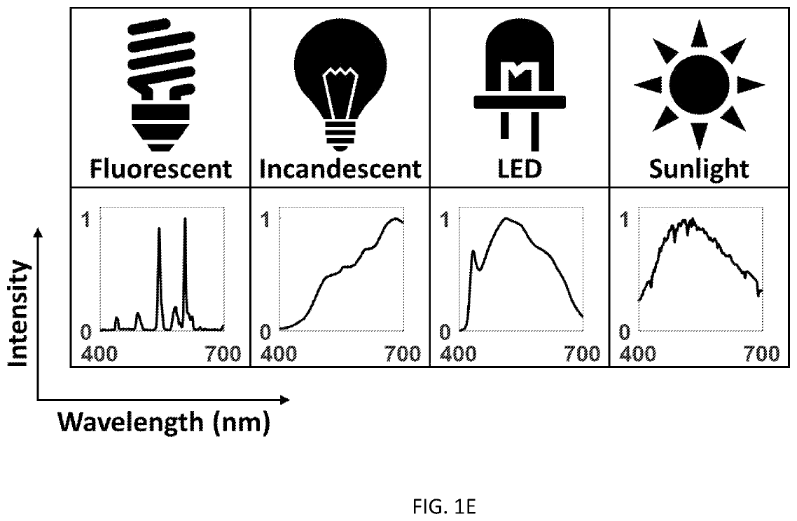

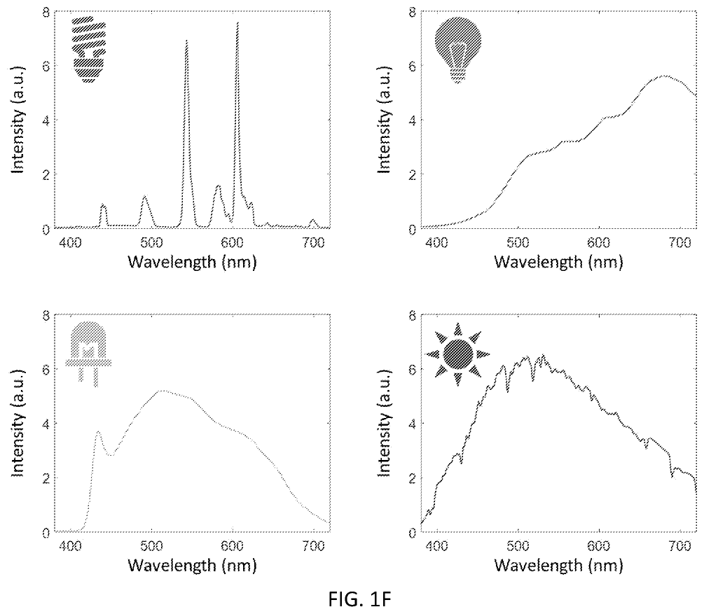

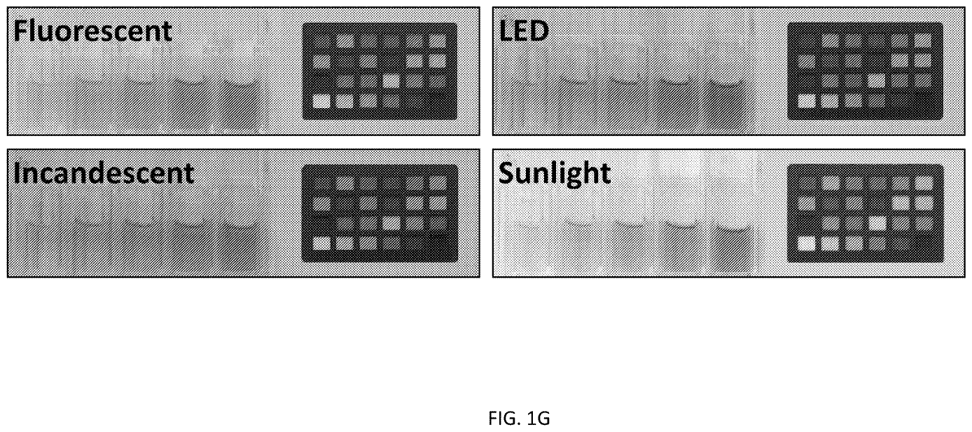

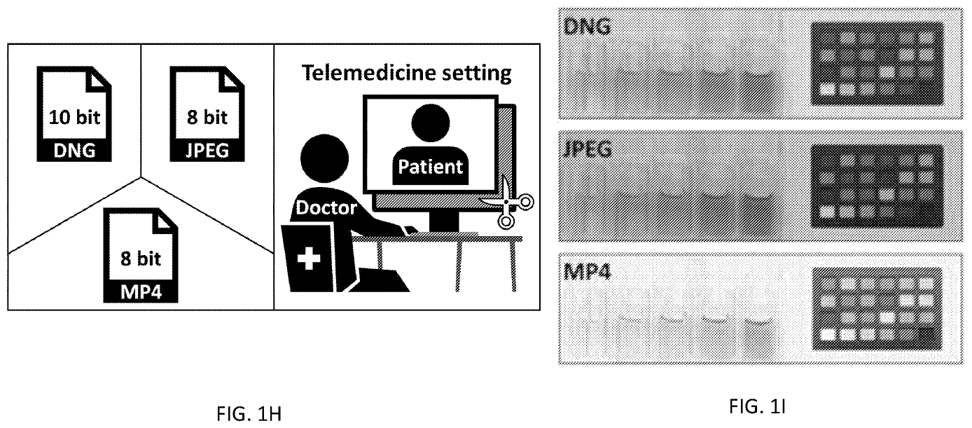

### DETAILED DESCRIPTION

For the purposes of promoting an understanding of the principles in the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

A novel method is disclosed herein that can use a ground truth color chart and correct colors in captured images to true colors. Towards this end, a machine learning methodology is provided that can be trained based on a ground truth color chart juxtaposed near an image of a tissue of interest against spectral and colorimetric information of the ground truth color chart. The machine learning methodology may be configured to receive inputs in one of many domains, including RGB, CIE XYZ, CIE RGB, CIE YUV, CIE UVW, CIE LAB, CIE LUV, HSL, HSV, HCL, and LMS; and then provide an output in the same or different domains selected from the above list of domains.

The ground truth color chart received by the method of the present disclosure incorporates a sufficient number of reference color patches thus allowing to use advanced machine learning techniques that can outperform conventional color correction computations. The machine learning approach of the present disclosure operates unlike a regression-based color correction computation methodology in which such methods are commonly used in machine vision systems and which require manual fine-tuning, depending on photo acquisition settings. In contrast, the present methodology offers a machine learning methodology that includes a training phase based on the ground truth color chart which is juxtaposed next to a scene or tissue of interest after which the trained setup can quickly and effectively correct colors in the remainder of the captured image in the above-described domains.

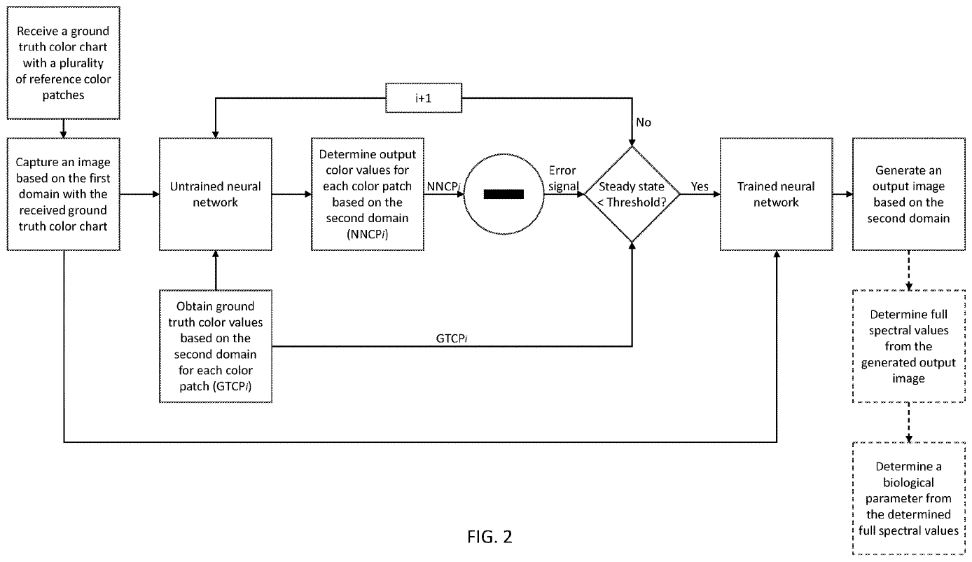

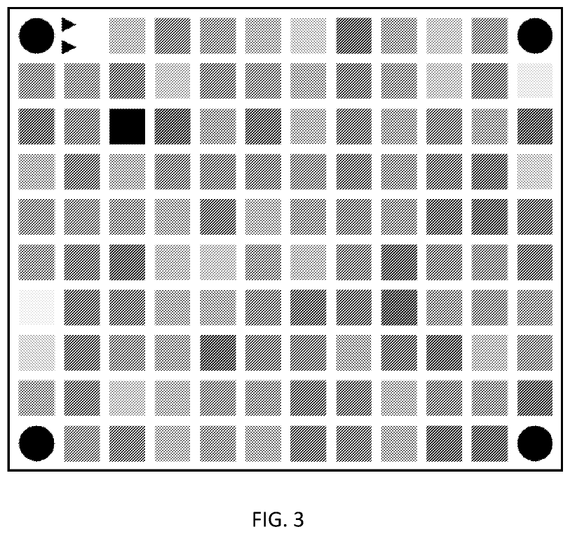

Thus, the present methodology describes a comprehensive method that can realize high fidelity of color accuracy in diagnostic photography through tissue-specific ground truth color chart and provide true color recovery. The methodology described herein is centered around peripheral perfusion and blood hemoglobin, however, the same methodology can be applied to any scene or tissue of interest. A color chart of biological tissue is thus provided that is highly sensitive and accurate to tissue and other biological content such as blood hemoglobin. After receiving the physical and spectral characterization of the ground truth color chart, the machine learning algorithm for gamut-learned color recovery is discussed using deep learning, which enables high fidelity of color accuracy in diverse settings. Using a series of diagnostic color scales, tissue phantoms, and whole blood-mimicking samples, we extensively compare the proposed method with the conventional regression-based color correction approaches. We further demonstrate the feasibility of an mHealth application that can noninvasively predict blood hemoglobin levels from single-shot photographs of easily accessible peripheral tissue across various photo acquisition settings. Referring to FIG. 2, a flowchart is presented that describes the methodology of the present disclosure at a high level. The flowchart in FIG. 2, thus represents a method for correcting colors in a captured image from a scene of interest. The method includes receiving a ground truth color chart having a plurality of reference color patches. An example of such a ground truth color chart is shown in FIG. 3. The method shown in FIG. 2 also includes capturing an image based on a first color domain from the scene of interest while having the received ground truth color chart juxtaposed with the scene of interest. Therefore, the captured image thereby includes the plurality of reference color patches. An example of such a scene of interest with the ground truth color chart juxtaposed therein is shown in FIG. 5. Next, the method shown in FIG. 2 indicates obtaining ground truth color values based on a second color domain for each of the reference color patches (hereinafter referred to as GTCPᵢ, where i has a value between 1 to n, where n represents the number of reference color patches). The ground truth color values may be provided (i.e., a priori known) or determined by applying the ground truth color chart to a spectrometer. The obtained ground truth color values of each color patch and the captured color patch are provided to an untrained neural network. A training phase of the neural network commences by using the plurality of reference color patches in the captured image and GTCPᵢ. The training of the neural network includes running an epoch for each of the color patches (i.e., i=1, 2, 3, . . . , N). For each iteration, the training phase includes inputting each of the captured plurality of reference color patches (first color domain) to the neural network at its input layer. The neural network then determines output color values for each of the plurality of color patches based on the second color domain (NNCPᵢ) and the associated input to the neural network. Next, the training phase involves establishing an error signal associated with a difference between NNCPᵢ and GTCPᵢ. This iterative process is repeated until the established error signal reaches a steady state below a threshold. Once the error signal reaches this state, the neural network is deemed as being trained. Next, the method as shown in FIG. 2 applies the trained neural network to the captured image to thereby generate an output image based on the second color domain with corrected colors.

In FIG. 2, according to one embodiment, the first color domain, is the same as the second color domain.

In FIG. 2, according to one embodiment, the first color domain, according to one embodiment, is different as the second color domain.

In FIG. 2, according to one embodiment, the scene of interest is from a biological tissue.

In FIG. 2, according to one embodiment, the tissue includes palpebral conjunctiva.

In FIG. 2, according to one embodiment, the tissue includes skin.

In FIG. 2, according to one embodiment, the tissue includes teeth.

In FIG. 2, according to one embodiment, the tissue includes tongue.

In FIG. 2, according to one embodiment, the tissue includes eye.

In FIG. 2, according to one embodiment, the tissue includes bulbar conjunctiva.

In FIG. 2, according to one embodiment, the tissue includes urine.

In FIG. 2, according to one embodiment, the tissue includes whole blood.

In FIG. 2, according to one embodiment, the tissue includes eye tears.

In FIG. 2, according to one embodiment, the tissue includes nailbed

In FIG. 2, according to one embodiment, the tissue includes external eye.

In FIG. 2, according to one embodiment, the tissue includes saliva.

In FIG. 2, according to one embodiment, the tissue includes stool.

In FIG. 2, according to one embodiment, the tissue includes body fluid.

In FIG. 2, according to one embodiment, the tissue includes biological solutions.

In FIG. 2, according to one embodiment, the predetermined domain is RGB.

In FIG. 2, according to one embodiment, the first color domain is CIE XYZ.

In FIG. 2, according to one embodiment, the first color domain is CIE RGB.

In FIG. 2, according to one embodiment, the first color domain is CIE YUV.

In FIG. 2, according to one embodiment, the first color domain is CIE UVW.

In FIG. 2, according to one embodiment, the first color domain is CIE LAB.

In FIG. 2, according to one embodiment, the first color domain is CIE LUV.

In FIG. 2, according to one embodiment, the first color domain is HSL.

In FIG. 2, according to one embodiment, the first color domain is HSV.

In FIG. 2, according to one embodiment, the first color domain is HCL.

In FIG. 2, according to one embodiment, the first color domain is LMS.

In FIG. 2, according to one embodiment, the second color domain is CIE XYZ.

In FIG. 2, according to one embodiment, the second color domain is CIE RGB.

In FIG. 2, according to one embodiment, the second color domain is CIE YUV.

In FIG. 2, according to one embodiment, the second color domain is CIE UVW

In FIG. 2, according to one embodiment, the second color domain is CIE LAB.

In FIG. 2, according to one embodiment, the second color domain is CIE LUV.

In FIG. 2, according to one embodiment, the second color domain is HSL.

In FIG. 2, according to one embodiment, the second color domain is HSV.

In FIG. 2, according to one embodiment, the second color domain is HCL.

In FIG. 2, according to one embodiment, the second color domain is LMS.

In FIG. 2, according to one embodiment, the input layer of the neural network has three nodes of three-color components.

In FIG. 2, according to one embodiment, the output layer of the neural network has three nodes of three-color components.

In FIG. 2, according to one embodiment, the neural network has a hidden layer.

In FIG. 2, according to one embodiment, the hidden layer has a plurality of nodes to incorporate a 2^{nd} order of parameters having at least four of these terms:

$${D}_{11} , {D}_{12} , {D}_{13} , {D}_{11}^{2} , {D}_{12}^{2} , {D}_{13}^{2} , {D}_{11} {D}_{12} , {D}_{12} {D}_{13} , {D}_{13} {D}_{11} , \\ \sqrt{{D}_{11} {D}_{12}} , \sqrt{{D}_{12} {D}_{13}} , \sqrt{{D}_{13} {D}_{11}}$$

wherein D₁₁ represents first color value of the first color domain,
D₁₂ represents second color value of the first color domain,
and D₁₃ represents third color value of the first color domain.

In FIG. 2, according to one embodiment, the hidden layer employs one of sigmoid activation function and sigmoid linear unit activation function.

In FIG. 2, according to one embodiment, the hidden layer has a plurality of nodes to incorporate a 3^{rd} order of parameters having at least four of these terms:

$${D}_{11} , {D}_{12} , {D}_{13} , {D}_{11}^{2} , {D}_{12}^{2} , {D}_{13}^{2} , {D}_{11} {D}_{12} , {D}_{12} {D}_{13} , {D}_{13} {D}_{11} , {D}_{11}^{3} , {D}_{12}^{3} , \\ {D}_{13}^{3} , {D}_{11}^{2} {D}_{12} , {D}_{11} {D}_{12}^{2} , {D}_{12}^{2} {D}_{13} , {D}_{12} {D}_{13}^{2} , {D}_{13}^{2} {D}_{11} , {D}_{13} {D}_{11}^{2} , {D}_{11} {D}_{12} {D}_{13} , \\ \sqrt{{D}_{11} {D}_{12}} , \sqrt{{D}_{12} {D}_{13}} , \sqrt{{D}_{13} {D}_{11}} , \sqrt[3]{{D}_{11}^{2} {D}_{12}} , \sqrt[3]{{D}_{11} {D}_{12}^{2}} , \\ \sqrt[3]{{D}_{12}^{2} {D}_{13}} , \sqrt[3]{{D}_{12} {D}_{13}^{2}} , \sqrt[3]{{D}_{13}^{2} {D}_{11}} , \sqrt[3]{{D}_{13} {D}_{11}^{2}} , \sqrt[3]{{D}_{11} {D}_{12} {D}_{13}}$$

wherein D₁₁ represents first color value of the first color domain,
D₁₂ represents second color value of the first color domain,
and D₁₃ represents third color value of the first color domain.

In FIG. 2, according to one embodiment, the hidden layer employs one of sigmoid activation function and sigmoid linear unit activation function.

In FIG. 2, according to one embodiment, the hidden layer has a plurality of nodes to incorporate a 4th order of parameters having at least four of these terms:

$${D}_{11} , {D}_{12} , {D}_{13} , {D}_{11}^{2} , {D}_{12}^{2} , {D}_{13}^{2} , {D}_{11} {D}_{12} , {D}_{12} {D}_{13} , {D}_{13} {D}_{11} , {D}_{11}^{3} , {D}_{12}^{3} , {D}_{13}^{3} , \\ {D}_{11}^{2} {D}_{12} , {D}_{11} {D}_{12}^{2} , {D}_{12}^{2} {D}_{13} , {D}_{12} {D}_{13}^{2} , {D}_{13}^{2} {D}_{11} , {D}_{13} {D}_{11}^{2} , {D}_{11} {D}_{12} {D}_{13} , {D}_{11}^{4} , \\ {D}_{12}^{4} , {D}_{13}^{4} , {D}_{11}^{3} {D}_{12} , {D}_{11} {D}_{12}^{3} , {D}_{12}^{3} {D}_{13} , {D}_{12} {D}_{13}^{3} , {D}_{13}^{3} {D}_{11} , {D}_{13} {D}_{11}^{3} , {D}_{11}^{2} {D}_{12}^{2} , \\ {D}_{12}^{2} {D}_{13}^{2} , {D}_{13}^{2} {D}_{11}^{2} , {D}_{11}^{2} {D}_{12} {D}_{13} , {D}_{11} {D}_{12}^{2} {D}_{13} , {D}_{11} {D}_{12} {D}_{13}^{2} , \\ \sqrt{{D}_{11} {D}_{12}} , \sqrt{{D}_{12} {D}_{13}} , \sqrt{{D}_{13} {D}_{11}} , \sqrt[3]{{D}_{11}^{2} {D}_{12}} , \sqrt[3]{{D}_{11} {D}_{12}^{2}} , \sqrt[3]{{D}_{12}^{2} {D}_{13}} , \\ \sqrt[3]{{D}_{12} {D}_{13}^{2}} , \sqrt[3]{{D}_{13}^{2} {D}_{11}} , \sqrt[3]{{D}_{13} {D}_{11}^{2}} , \sqrt[3]{{D}_{11} {D}_{12} {D}_{13}} , \sqrt[4]{{D}_{11}^{3} {D}_{12}} , \sqrt[4]{{D}_{11} {D}_{12}^{3}} , \\ \sqrt[4]{{D}_{12}^{3} {D}_{13}} , \sqrt[4]{{D}_{12} {D}_{13}^{3}} , \sqrt[4]{{D}_{13}^{3} {D}_{11}} , \sqrt[4]{{D}_{13} {D}_{11}^{3}} , \sqrt[4]{{D}_{11}^{2} {D}_{12}^{2}} , \sqrt[4]{{D}_{12}^{2} {D}_{13}^{2}} , \\ \sqrt[4]{{D}_{13}^{2} {D}_{11}^{2}} , \sqrt[4]{{D}_{11}^{2} {D}_{12} {D}_{13}} , \sqrt[4]{{D}_{11} {D}_{12}^{2} {D}_{13}} , \sqrt[4]{{D}_{11} {D}_{12} {D}_{13}^{2}}$$

wherein D₁₁ represents first color value of the first color domain,
D₁₂ represents second color value of the first color domain,
and D₁₃ represents third color value of the first color domain.

In FIG. 2, according to one embodiment, the hidden layer employs one of sigmoid activation function and sigmoid linear unit activation function.

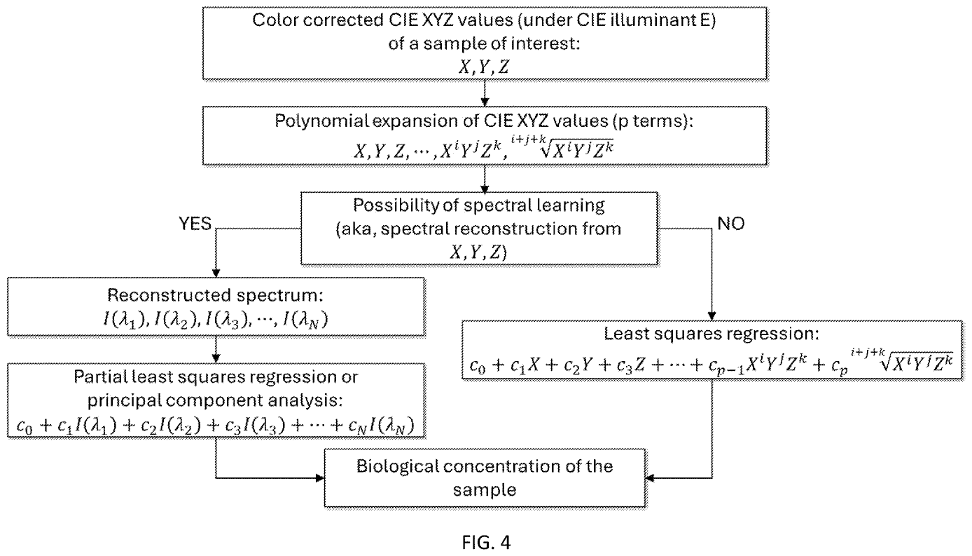

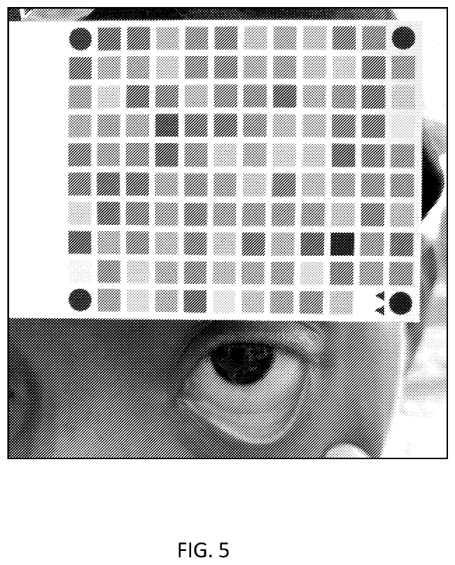

So far, the present disclosure has described color correction based on a ground truth color chart. However, once colors are corrected in an image, the present disclosure provides a process by which concentration of a substance can be ascertained from an image based on the flowchart provided in FIG. 4. This figure shows the steps for the use of color-corrected color values of a sample of interest (not from the ground truth color chart) for colorimetric diagnostic applications. Accordingly, two different approaches are described for obtaining the biological concentration of the sample using color-corrected color values in the second color domain (e.g. CIE XYZ values under CIE Illuminant E). In both cases, color corrected CIE XYZ values of the sample serve as a frontend input to achieve diagnostic color accuracy with a single-shot photo. Color corrected CIE XYZ values of the sample allows achieving diagnostic-level color accuracy, which is not affected by ambient light conditions or smartphone/device models. Accordingly, the present disclosure provides steps when the spectral learning (i.e., spectral reconstruction) is possible, such that the concentration of the sample can be computed by two steps: 1) spectral learning and 2) partial least squares regression (or principal component analysis).

In the case of spectral learning, color corrected CIE XYZ values of the sample are used to reconstruct the full spectral information from the color corrected CIE XYZ values. Spectral learning addresses an ill-posed challenge. Depending on the availability of existing data, several machine learning approaches can be used for solving this underdetermined problem including: compressed sensing or sparsity regularization (L1 norm minimization), least squares regression (L2 norm minimization), and deep learning. Among these approaches, statistical learning using fixed-design linear regression with polynomial expansions offers a highly stable inverse calculation that can transform CIE XYZ values to high-resolution spectral data owing to the nature of L2 norm minimization.

In the first approach of spectral learning with CIE XYZ values the full spectral values from the tristimulus color values of the sample are determined in a second color domain (e.g. the color corrected CIE XYZ values of the sample), when relevant spectral data are available. The full spectral values are obtained from:

$${B}_{N \times r} = {R}_{N \times p} {A}_{p \times r} ,$$

wherein N represents number of wavelengths of spectral values based on a predetermined resolution,
r represents the number of existing spectral data,
R_{N×p} represents a transformation matrix to transform the tristimulus color values of the sample to full spectral values, where p represents number of polynomial expansion terms, and
A_{p×r} represents an expanded the tristimulus color values of the sample represented by:

$${A}_{p \times r} = {[ \begin{matrix}{D}_{{21}_{1}} & {D}_{2 {2}_{1}} & {D}_{2 {3}_{1}} & ⋯ & {D}_{2 {1}_{1}}^{i} {D}_{2 {2}_{1}}^{j} {D}_{2 {3}_{1}}^{k} & \sqrt[i + j + k]{{D}_{{21}_{1}}^{i} {D}_{2 {2}_{1}}^{j} {D}_{2 {3}_{1}}^{k}} \\ ⋮ & ⋮ & ⋮ & ⋮ & ⋮ & ⋮ \\ {D}_{2 {1}_{r}} & {D}_{2 {2}_{r}} & {D}_{2 {3}_{r}} & … & {D}_{{21}_{r}^{i}} {D}_{2 {2}_{r}}^{j} {D}_{2 {3}_{r}}^{k} & \sqrt[i + j + k]{{D}_{21 r}^{i} {D}_{22 r}^{j} {D}_{2 {3}_{r}}^{k}}\end{matrix} ]}^{T}$$

wherein i, j, and k represent degree of expansion,
D₂₁ represents the first color value (the color corrected CIE X values of the sample) of the second color domain,
D₂₂ represents the second color value (the color corrected CIE Y values of the sample) of the second color domain,
and D₂₃ represents the third color (the color corrected CIE Z values of the sample) of the second color domain, the second color domain can be CIE XYZ. This method allows determining a biological concentration from the determined full spectral data of the sample in the second color domain. In this method the biological concentration may be blood hemoglobin and which is determined based on least partial least squares regression, based on the determined spectral values as:

$$Hgb = {c}_{0} + {c}_{1} I ( {λ}_{1} ) + {c}_{2} I ( {λ}_{2} ) + {c}_{3} I ( {λ}_{3} ) … + {c}_{N} I ( {λ}_{N} )$$

wherein Hgb represents the concentration of blood hemoglobin,
coefficients c₀, c₁, c₂, c₃, . . . , c_{N} are determined using a priori subject population blood hemoglobin concentration data vs. spectral intensity data across a range of wavelengths (λ₁, λ₂, λ₃ . . . λ_{N}).

However, if spectral learning (i.e., spectral reconstruction) is not possible, the concentration of the sample can be computed directly by using the CIE XYZ values of the sample.

Typically, this is the case where reference color patches in the color chart (e.g. urine strips or finger prick strips) contain the corresponding concentration information of interest to estimate. Color-corrected CIE XYZ values of the sample of interest, juxtaposed with the color chart, can be utilized to return the concentration of the sample. In such instances, a mathematical model can be directly formulated to compute the concentration, using the CIE XYZ values of the sample. Thus, this method includes determining concentrations of the sample compared with reference color patches in a color chart (e.g. existing color chart for urine strips or finger prick strips), from the tristimulus color values of the sample in the second color domain. In this method, the second color domain can be CIE XYZ. This method may also include determining a biological concentration from the determined color values of the sample in the second color domain (e.g., the color-corrected CIE XYZ values of the sample). Accordingly, the biological parameter can be the sample concentration determined based on a least squares regression that is based on a polynomial expansion of the determined color values of the sample expressed as:

$$Concentration = {c}_{0} + {c}_{1} {D}_{21} + {c}_{2} {D}_{22} + {c}_{3} {D}_{23} … + {c}_{p - 1} {D}_{21}^{i} {D}_{22}^{j} {D}_{23}^{k} + {c}_{p} \sqrt[i + j + k]{{D}_{21}^{i} {D}_{22}^{j} {D}_{23}^{k}}$$

wherein i, j, and k represent degree of expansion,
D₂₁ represents the first color value (the color corrected CIE X values of the sample) of the second color domain,
D₂₂ represents the second color value (the color corrected CIE Y values of the sample) of the second color domain,
and D₂₃ represents the third color (the color corrected CIE Z values of the sample) of the second color domain,
p represents number of polynomial expansion terms,
and coefficients c₀, c₁, c₂, c₃, . . . , c_{N} are determined from the determined color values and associated concentrations of the reference color patches in the existing color chart (e.g. urine strips or finger prick strips).

We apply the gamut-learned color recovery method to a clinical laboratory application. The color-corrected values serve as the frontend input of a diagnostic algorithm to compute a concentration of interest. As a test model system of relying on peripheral tissue, we take advantage of an mHealth application that noninvasively predicts blood hemoglobin levels from digital photos of the palpebral conjunctiva. We analyze a pilot human study data (clinicaltrials.gov NCT05045820) that aims to improve the quality of sickle cell disease (SCD) patient care by minimizing iatrogenic blood losses caused by frequent blood draws. The clinical data include photos of the palpebral conjunctiva with the ground truth color chart and clinical laboratory blood hemoglobin results of venous blood draws as the gold standard, from fifteen SCD patients aged from 14 to 73 years (FIG. 5).

We focus on reproducibility (inter-measurement reliability) that is a variation among different users, devices, and settings, when the same patient is assessed. The clinical photos are acquired under mixed light conditions of sunlight, fluorescent tube, and white-light LED in indoor settings (FIG. 5), immediately before or after venous blood draws. For inter-blood hemoglobin assessments (reproducibility), we employ a substantial dataset of 156 photos from both the left and right eyelids in the RAW (i.e., DNG) and JPEG file formats, using SAMSUNG GALAXY S21 and APPLE IPHONE 12PRO.

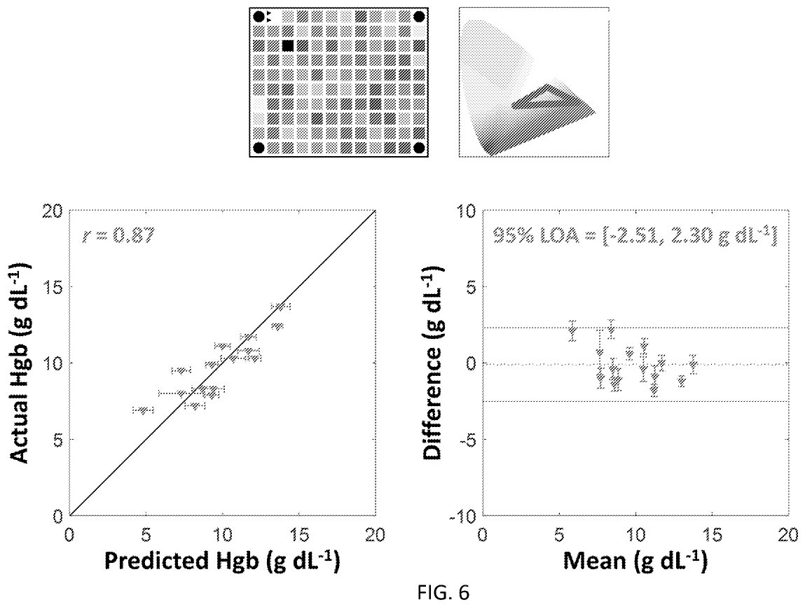

FIG. 6 and TABLE 1 show the blood hemoglobin quantification results using the ground truth color chart. The scatter plot and the error bar represent the mean and the standard deviation, respectively. The linear correlation between the computed blood hemoglobin levels and clinical laboratory results returns a high correlation coefficient of 0.87 with narrow 95% limits of agreement (95% LOA) of [−2.51, 2.30 g dL⁻¹] and bias of −0.11 g dL⁻¹ in the Bland-Altman plot. Importantly, the ranges of 95% LOA are highly comparable to those of capillary blood sampling (e.g., finger prick) tests. A pooled standard deviation of the computed blood hemoglobin levels captures the reproducibility, returning a value of 1.26 g dL⁻¹. Overall, these results demonstrate that the proposed gamut-learned color recovery method enables simple, reliable, consistent, and reproducible color accuracy for diagnostic bioassays and bioimaging.

TABLE 1
Inter-blood hemoglobin quantification results

| | Samsung Galaxy S21 | | | | | | | | Apple iphone 12 Pro | | | | | | Venous |
|---|---|---|---|---|---|---|---|---|---|---|---|---|---|---|---|
| | DNG (RAW) | | | | JPEG | | | | DNG (RAW) | | | | Computed Hgb | | blood draw: |
| | Left | | | | | | | | | | | | (g dL⁻¹) | | the gold |
| Patient | Photo | Photo | Right | | Left | | Right | | Left | | Right | | | Standard | standard |
| # | #1 | #2 | #1 | #2 | #1 | #2 | #1 | #2 | #1 | #2 | #1 | #2 | Mean | deviation | (g dL⁻¹) |
| 1 | 9.4 | 9.3 | 10.8 | 10.0 | 11.9 | 11.4 | 13.8 | 12.6 | 10.0 | 9.8 | 13.4 | 13.2 | 11.3 | 1.6 | 10.3 |
| 2 | 14.3 | 14.1 | 11.3 | 12.8 | | 13.9 | 12.1 | | 11.8 | 11.4 | 10.8 | 10.5 | 12.3 | 1.4 | 10.8 |
| 3 | 9.2 | 9.6 | | 4.1 | 9.8 | 10.4 | | 4.4 | | | | | 7.9 | 2.9 | 8.0 |
| 4 | 5.2 | 5.6 | 7.0 | 6.1 | 4.2 | 5.7 | 7.2 | 6.5 | 4.0 | 4.3 | 3.2 | 5.8 | 5.4 | 1.3 | 6.9 |
| 5 | 10.5 | 10.1 | 10.3 | 11.4 | 11.5 | 11.0 | 11.2 | 12.4 | 9.9 | 10.0 | 9.6 | 8.9 | 10.6 | 1.0 | 11.1 |
| 6 | 8.7 | 9.0 | 10.5 | 9.6 | 9.2 | 9.7 | 10.7 | 9.4 | 9.4 | 9.8 | 11.1 | 11.1 | 9.9 | 0.8 | 9.9 |
| 7 | 13.3 | 13.4 | 14.0 | 13.9 | 14.5 | 14.8 | 14.9 | 15.0 | 15.3 | 14.7 | 13.6 | 13.5 | 14.2 | 0.7 | 12.4 |
| 8 | 7.8 | 6.8 | | 10.4 | 9.1 | 8.1 | | | 9.5 | 7.4 | 10.4 | 9.5 | 8.8 | 1.3 | 7.2 |
| 9 | 9.6 | 11.5 | 7.1 | 9.0 | 10.5 | | 10.5 | | 7.9 | 8.5 | 9.2 | | 9.3 | 1.4 | 8.3 |
| 10 | | | 8.5 | 9.0 | | 5.7 | 9.1 | 8.3 | | | 7.5 | 7.2 | 7.9 | 1.2 | 9.5 |
| 11 | 9.4 | 9.5 | 10.6 | 11.6 | 10.1 | 10.2 | | | 8.2 | 9.0 | 10.2 | 10.2 | 9.9 | 0.9 | 7.9 |
| 12 | 8.9 | 10.3 | 11.6 | 12.2 | 8.4 | 9.3 | 9.4 | | 8.0 | 9.5 | 10.5 | 11.8 | 10.0 | 1.4 | 8.3 |
| 13 | 13.5 | 13.5 | 12.4 | 12.7 | 12.4 | 12.5 | 13.3 | 13.1 | 12.8 | 13.2 | 10.6 | 12.3 | 12.7 | 0.8 | 10.3 |
| 14 | 12.2 | 12.0 | 10.7 | 11.0 | 13.9 | 13.8 | 12.8 | 13.0 | 12.6 | 12.3 | 11.3 | 11.4 | 12.3 | 1.0 | 11.7 |
| 15 | 13.5 | 13.7 | 14.6 | 14.8 | 15.8 | 16.6 | | | 13.1 | 12.7 | 14.4 | 14.7 | 14.4 | 1.2 | 13.7 |

As a non-parametric statistical analysis, we conducted Bland-Altman plots to compare the mHealth blood hemoglobin assessments with the ground truth color chart and clinical laboratory blood hemoglobin tests (i.e., venous blood draws). Bland-Altman analyses are extensively performed to evaluate the level of agreement between two different sets of measurements or readings. The bias was defined by a mean of differences between the computed blood hemoglobin levels using the ground truth color chart and the clinical laboratory blood hemoglobin levels (d=Hgb^{Color chart}−Hgb^{Clinical}):

$$Bias = \overline{d} = \frac{1}{n} {\sum }_{t = 1}^{n} {d}_{t}$$

where n is the number of patients. The 95% limits of agreement (95% LOA) was defined by a 95% prediction interval of the standard deviation:

$$95 \% LOA = \overline{d} \pm 1 \text{.96} \sqrt{\frac{1}{n - 1} {\sum }_{t = 1}^{n} {( {d}_{t} - \overline{d} )}^{2}}$$

When reference color patches in a color chart for urine strips or finger prick strips contain corresponding concentration information of interest to estimate, color-corrected CIE XYZ values (input) derived from the sample of interest, juxtaposed with the color chart, can be utilized to return the concentration of the sample (output). In such instances, a mathematical model can be formulated to predict the concentration (output), using the CIE XYZ values (input).

The World Health Organization (WHO) Haemoglobin Colour Scale (or Tallquist Haemoglobin Scale) is a diagnostic method for estimating blood hemoglobin concentration from a drop of blood. It comprises six reference color patches representing blood hemoglobin levels at 4.7, 6.3, 7.8, 9.4, 10.9, 12.5, 14.1, and 15.6 g dL⁻¹. A drop of blood smeared onto filter paper is compared with the WHO Haemoglobin Colour Scale. Estimation is achieved by matching the blood sample with the color standards through circular apertures placed in the center of each color standard. However, the original clinical utility of this method is limited due to the error-prone subjective nature of color comparisons made with the naked eye.

After capturing a photograph of the blood sample juxtaposed with the WHO Haemoglobin Colour Scale, the CIE XYZ values of the blood sample under CIE illuminant E are recovered using the color chart. These color-corrected values serve as the frontend input of an algorithm to compute the blood hemoglobin level.

Those having ordinary skill in the art will recognize that numerous modifications can be made to the specific implementations described above. The implementations should not be limited to the particular limitations described. Other implementations may be possible.

1. A method for correcting colors in a captured image, comprising:
receiving a ground truth color chart having a plurality of reference color patches;
capturing an image based on a first color domain from a scene or sample of interest juxtaposed with the received ground truth color chart, the captured image thereby including the plurality of reference color patches;
obtaining ground truth color values associated with each of the reference color patches in a second color domain;
generating a scene or sample-specific dataset using the plurality of color values of the reference color patches in the ground truth color chart in the first color domain and the second color domain as the input and output, respectively;
training a neural network of color correction with the scene or sample-specific dataset; and
applying the trained neural network to convert portion of the scene or sample of interest outside the ground truth color chart in the captured image in the first color domain to thereby generate an output image based on the second color domain.

2. The method of claim 1, wherein the first color domain is the same or different than the second color domain.

3. (canceled)

4. The method of claim 1, wherein the ground truth color values based on the second color domain associated with each of the reference color patches are defined under a standard illuminant established by the International Commission on Illumination (CIE).

5. The method of claim 1, wherein spatial nonuniformity of illumination is corrected in the capture image.

6. (canceled)

7. (canceled)

8. The method of claim 1, wherein the biological tissue includes at least one of palpebral conjunctiva, skin, wound, teeth, tongue, eye, bulbar conjunctiva, urine, whole blood, eye tears, nailbed, external tissue surrounding eye, stool, saliva, body fluid, and biological solutions.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The method of claim 1, wherein each of the first or second color domains includes at least one of RGB, CIE XYZ, CIE YUV, CIE UVW, CIE RGB, CIE LAB, CIE LCH, CIE LUV, HSL, HSV, HCL, LMS, OKLAB, OKLCH, and YCbCr.

23. (canceled)

24. (canceled)

25. (canceled)

26. (canceled)

27. (canceled)

28. (canceled)

29. (canceled)

30. (canceled)

31. (canceled)

32. (canceled)

33. The method of claim 1, wherein the neural network is a multilayer perceptron comprising one or more hidden layers that employ one or more activation functions, wherein the neural network includes connections defined by weights that are initialized by sampling from a predetermined distribution.

34. (canceled)

35. (canceled)

36. A method of determining concentration of a molecule in a sample from a captured image, comprising:
establishing a color-corrected image (output image) from a captured image from a scene or sample of interest, the output image provides color corrections from a first color domain to a second color domain, the established output image associated with color values of the sample in the second color domain; and
using color values of the output image of the sample in the second color domain to determine the concentration of a molecule of interest.

37. The method of claim 36, further comprising:
determining full spectral values from the associated color values of the output image in the second color domain, when a priori spectral data are available.

38. The method of claim 37, wherein the full spectral values are calculated from
B_{N×r}=R_{N×p}A_{p×r},
wherein N represents number of wavelengths of spectral values based on a predetermined resolution,
r represents number of a priori spectral data,
R_{N×p} represents a transformation matrix to transform the determined color values of the output image in the second color domain to full spectral values, where p represents number of polynomial expansion terms, and
A_{p×r} represents an expanded matrix of the determined color values of the output image in the second color domain represented by:
$${A}_{p \times r} = {[ \begin{matrix}{D}_{{21}_{1}} & {D}_{2 {2}_{1}} & {D}_{2 {3}_{1}} & ⋯ & {D}_{2 {1}_{1}}^{i} {D}_{2 {2}_{1}}^{j} {D}_{2 {3}_{1}}^{k} & \sqrt[i + j + k]{{D}_{{21}_{1}}^{i} {D}_{2 {2}_{1}}^{j} {D}_{2 {3}_{1}}^{k}} \\ ⋮ & ⋮ & ⋮ & ⋮ & ⋮ & ⋮ \\ {D}_{2 {1}_{r}} & {D}_{2 {2}_{r}} & {D}_{2 {3}_{r}} & … & {D}_{{21}_{r}^{i}} {D}_{2 {2}_{r}}^{j} {D}_{2 {3}_{r}}^{k} & \sqrt[i + j + k]{{D}_{21 r}^{i} {D}_{22 r}^{j} {D}_{2 {3}_{r}}^{k}}\end{matrix} ]}^{T}$$
wherein i, j, and k represent degree of expansion,
D₂₁ represents first color value of the second color domain,
D₂₂ represents second color value of the second color domain,
and D₂₃ represents third color value of the second color domain.

39. The method of claim 36, wherein the determined concentration of the molecule of interest establishes blood hemoglobin.

40. The method of claim 39, wherein blood hemoglobin concentration is determined based on the calculated full spectral values and a spectrum-to-concentration model as compared to a predetermined population spectral values and associated population blood hemoglobin concentration values using partial least squares regression.

41. The method of claim 40, wherein the spectrum-to-concentration model is:
$$Hgb = {c}_{0} + {c}_{1} I ( {λ}_{1} ) + {c}_{2} I ( {λ}_{2} ) + {c}_{3} I ( {λ}_{3} ) … + {c}_{N} I ( {λ}_{N} )$$
wherein Hgb represents blood hemoglobin concentration, and
coefficients c₀, c₁, c₂, c₃, . . . , c_{N} are determined using a priori subject population blood hemoglobin concentration data vs. spectral intensity data across a range of wavelengths (λ₁, λ₂, λ₃, . . . , λ_{N}).

42. The method of claim 36, further comprising:
receiving a color-to-concentration model expressing concentration of the molecule based on colors in the second color domain using least squares regression; and
applying the color-to-concentration model to the output image to thereby obtain concentration of the molecule from the output image.

43. The method of claim 42, wherein the color-to-concentration model is:
$$Concentration = {c}_{0} + {c}_{1} {D}_{21} + {c}_{2} {D}_{22} + {c}_{3} {D}_{23} … + {c}_{p - 1} {D}_{21}^{i} {D}_{22}^{j} {D}_{23}^{k} + {c}_{p} \sqrt[i + j + k]{{D}_{21}^{i} {D}_{22}^{j} {D}_{23}^{k}}$$
wherein i, j, and k represent degree of expansion,
D₂₁ represents the first color value of the second color domain,
D₂₂ represents the second color value of the second color domain,
D₂₃ represents the third color value of the second color domain,
p represents number of polynomial expansion terms, and
coefficients c₀, c₁, c₂, c₃, . . . , c_{N} are determined using a priori subject population molecule concentration data vs. color values in the second color domain.

44. The method of claim 36, wherein the first color domain is the same as the second color domain or different than the second color domain.

45. (canceled)

46. The method of claim 36, wherein the second color domain is defined under a standard illuminant established by the International Commission on Illumination (CIE) illuminant E.

47. (canceled)

48. The method of claim 36, wherein the biological tissue includes at least one of palpebral conjunctiva, skin, teeth, wound, tongue, eye, bulbar conjunctiva, urine, whole blood, eye tears, nailbed, external tissue surrounding eye, saliva, stool, external body fluid, and biological solutions.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The method of claim 36, wherein each of the first or second color domains includes at least one of RGB, CIE XYZ, CIE YUV, CIE UVW, CIE RGB, CIE LAB, CIE LCH, CIE LUV, HSL, HSV, HCL, LMS, OKLAB, OKLCH, and YCbCr.

64. (canceled)

65. (canceled)

66. (canceled)

67. (canceled)

68. (canceled)

69. (canceled)

70. (canceled)

71. (canceled)

72. (canceled)

73. (canceled)

74. The method of claim 1, wherein the neural network is implemented by image-wide row-by-row processing.
